(19) United States
(12) Patent Application Publication
(10) Pub. No.: US 2025/0066280 A1
(43) Pub. Date: Feb. 27, 2025

(54) UNREFINED NEODECANOIC ACID SOURCE AS A RAW MATERIAL FOR H2S SCAVENGING METAL CARBOXYLATE

(71) Applicant: Heritage Research Group, LLC, Indianapolis, IN (US)

(72) Inventor: Ross Poland, Indianapolis, IN (US)

(73) Assignee: Heritage Research Group, LLC, Indianapolis, IN (US)

(21) Appl. No.: 18/945,921

(22) Filed: Nov. 13, 2024

Related U.S. Application Data
(63) Continuation of application No. PCT/CN2023/067038, filed on May 16, 2023.
(60) Provisional application No. 63/364,747, filed on May 16, 2022.

(51) Int. Cl. C07C 51/41

(52) U.S. Cl. CPC C07C 51/41

(57) ABSTRACT

The present disclosure includes a process for isolating the carboxylic acids portion of a material stream and reacting it with a metal source to product a blend of metal carboxylate species which are anticipated to be effective H₂S scavenging chemicals. In one embodiment, an “unrefined” or minimally refined acid stream in the production of a metal carboxylate-based H₂S scavenger, which is very attractive to use because the resultant metal carboxylate product will have essentially identical hydrogen sulfide scavenging capabilities as an analogous “pure” metal carboxylates while retaining the requisite oil solubility and stability.

### CROSS REFERENCE TO RELATED APPLICATION

This application continuation of International Application No. PCT/US2023/067038 filed May 16, 2023, which claims the benefit of U.S. Provisional Application No. 63/364,747 filed May 16, 2022, each of which are hereby incorporated herein by reference in their entirety.

### BACKGROUND

Hydrogen sulfide is a poisonous, invisible, naturally occurring gas produced by the degradation of sulfur-containing organic compounds. It is immediately dangerous to humans at concentrations as low as 100 ppm, and can be immediately fatal above 500 ppm. OSHA regulates H2S exposure to a 10 ppm per 8 hour time-weighted average with a short-term exposure limit of 15 ppm. It is present in many areas of industry, particularly in oil and gas. Certain crude slates are predisposed to generate high amounts of H2S, which can concentrate enough within closed spaces and in or around storage/transit containers that it becomes an acute safety hazard. Despite smelling distinctly of rotten eggs in non-lethal concentrations, at high enough concentrations it can deaden olfactory receptors almost immediately, making dangerous concentrations nearly impossible to detect without the use of monitoring equipment. H2S exposure results in many personnel incidents, which in rare cases can be fatal, so awareness and mitigation of H2S is a critical component of workplace safety.

In particular, H2S safety is important when working with asphalt. Asphalt can contain very high amounts of H2S, especially if the producing refinery tends to process a sour crude slate. It is important to be vigilant of H2S when working with asphalt for several reasons: the refining process concentrates heavy aromatic species containing sulfur linkages, the high latent storage temperature of asphalt is enough to produce H2S in a process known as thermal cracking, and high temperature and viscosity causes H2S latent in asphalt liquid to partition into gas phase rather than remain in liquid. H2S can concentrate in the gas headspace at the top of storage tanks or shipping containers (for example, in trucks or railcars), and as operators open these containers they can inadvertently be exposed to large, potentially fatal concentrations of H2S.

A common mitigation route to mitigating the H2S risk of working with potentially H2S-containing organic material is to pacify the H2S content through use of a chemical scavenger. Chemical scavengers work by preferentially reacting with H2S on a molecular level to convert it to a non-dangerous form. This concept, by utilizing various types of chemistry, can be applied to any hydrocarbon or aqueous stream. For asphalt, the most common method of H2S scavenging is to blend a metal-based complex into the asphalt which converts the hydrogen sulfide to a solid metal sulfide, which is nonvolatile, thermally stable, and poses no health risk to workers in the vicinity of the asphalt. The scavenger proceeds by the following general reaction scheme (I):

M(L)ₓ+H₂S->MS+X HL (I)

Scavenging can be accomplished by adding a metal powder such as iron or zinc oxide, but most commonly is done by adding the metal in a liquid form as a slurried oxide or carbonate or an oil-soluble metal carboxylate complex dissolved in a compatible solvent system. The most commonly used, commercially available metal-based scavenger is zinc octoate. More specifically, the “octoate” refers to a branched isomer of octanoic acid, 2-ethyhexanoic acid. This additive is a very effective H₂S scavenger—it reacts quickly, can absorb high H₂S per pound scavenger, and is easy to store and apply owing to its excellent physical properties.

Therefore, there remains a need for improved scavenging of H₂S and reducing the risks of exposure to the same.

### BRIEF SUMMARY OF THE INVENTION

In certain aspects, the present disclosure includes methods and compositions of matter useful for mitigation and/or scavenging of H₂S.

In further aspects, the present disclosure includes methods for preparing an oil-soluble metal carboxylate. In certain aspects, a carboxylate stream is reacted with a metal oxide. In still further aspects, the carboxylate stream comprises C5-C9 carboxylic acids, C10 carboxylic acids, and/or C11+ carboxylic acids (carboxylic acids with 11 or more carbon atoms). In some aspects, the C5-C9 carboxylic acids may comprise a C5 carboxylic acid, a C6 carboxylic acid, a C7 carboxylic acid, a C8 carboxylic acid, and/or a C9 carboxylic acid. In certain aspects C5-C9 carboxylic acids may comprise about 0.1 percent by weight (wt. %) to about 1 percent by weight (wt. %) of the carboxylate stream. In some aspects, C11+ carboxylic acids may comprise less than 10 percent by weight (wt. %) of the carboxylate stream such as about 5 percent by weight (wt. %) to about 10 percent by weight (wt. %), about 1 to about 10 wt. %, or about 1 to about 5 wt. % of the carboxylate stream. In still other aspects, C10 carboxylic acids may comprise ab out 75 percent by weight (wt. %) to about 95 percent by weight (wt. %) of the carboxylate stream.

In some aspects, a carboxylate stream may be prepared by a distillation, for example, but not limited to an azeotropic distillation. In some aspects, an azeotropic distillation may be performed with water. In certain aspects, a carboxylate stream may comprise non-acidic components. In certain aspects, non-acidic components may comprise about 0.1 percent by weight (wt. %) to about 5 percent by weight (wt. %) of the carboxylate stream and/or non-acidic components may comprise about 0.5 percent by weight (wt. %) to about 3 percent by weight (wt. %) of the carboxylate stream. In other aspects, non-acidic components of the carboxylate stream may include, but are not limited to, aldehydes and/or ethers.

In certain aspects a metal oxide may be prepared. In some embodiments, the metal oxide comprises zinc oxide. Other metals that may be used in various embodiments of the present disclosure include, but are not limited to iron and copper, for example, an oxide of iron and/or an oxide of copper. In some embodiments, more than one metal may be used, such as a combination of an oxide of zinc, an oxide of iron, and/or an oxide of copper.

Other aspects and features of the disclosed invention are discussed throughout the specification.

### BRIEF DESCRIPTION OF THE DRAWINGS

FIG. 1 shows a Liquid Chromatography Mass Spec (“LCMS”) analysis for two samples of the waste stream distilled to 100° C.

### DETAILED DESCRIPTION:

For the purposes of promoting an understanding of the principles of the invention, reference will now be made to certain embodiments and specific language will be used to describe the same. It will nevertheless be understood that no limitation of the scope of the invention is thereby intended, such alterations and further modifications, and such further applications of the principles of the invention as described herein being contemplated as would normally occur to one skilled in the art to which the invention relates. Additionally, in the detailed description below, numerous alternatives are given for various features. It will be understood that each such disclosed alternative, or combinations of such alternatives, can be combined with the more generalized features discussed in the Summary above, or set forth in the embodiments described below to provide additional disclosed embodiments herein.

A novel neodecanoic acid production process's distillation byproduct stream (for example, but not limited to a waste stream) has been identified as a source for producing an H₂S-scavenging material. Analysis of this stream indicated it comprises a mixture of roughly one-third (⅓) low-flash, light isoparaffinic hydrocarbon, and two-thirds (⅔) a mixture of carboxylic acids, with a carbon number ranging from 5 to as high as 13. In the present disclosure, we surprisingly discovered a purification method via azeotropic co-distillation of the lower-boiling point components of the waste stream with water, which selectively removes the low-flash isoparaffins (those with a flash point below 140° F.) and sub-7 carbon acids, leaving behind a >90% purity neodecanoic acid material, with the remaining acid being carboxylic acids of similar carbon number. In certain embodiments this material, though an impure neodecanoic acid stream, may be an ideal starting material for an oil-soluble metal carboxylate complex that can be used as an asphalt H₂S scavenger.

TABLE 1
Neo Lights Stream Composition

| Component | Mass % |
|---|---|
| Very light hydrocarbons, possibly | 12% |
| butane/hexane and other low MW oxygenates | |
| not captured with cool water condenser | |
| during 100° C. distillation | |
| Material recovered from condenser after | 11% |
| 100° C. distillation for ~2 hours recovered in | |
| cool water condenser | |
| Distillation bottoms - primarily C8-C10 | 67% |
| carboxylic acid | |

TABLE 2
LCMS Analysis of Waste Stream Composition

| | Crude Neo | Crude Neo |
|---|---|---|
| | Lights Stream* | Lights** |
| | (before 100° C. | (bottoms after |
| Component | distill.) | 100° C. distill.) |
| Unknown polar components | 4% | 2% |
| (Mass %) | | |
| (aldehydes and/or ethers, and | | |
| other low MW non-acidic | | |
| compounds) | | |
| C5-C9 Neoacids (Mass %) | <1% | <1% |
| Neodecanoic acids | 74% | 90% |
| (C₁₀H₂₀O₂) (likely contains | | |
| portions of other MWs too) | | |
| C₁₁H₂₁O₂, C₁₂H₂₄O₂, | 22% | 8% |
| C₁₃H₂₆O₂, & other High | | |
| molecular weight products | | |
| (Mass %) | | |

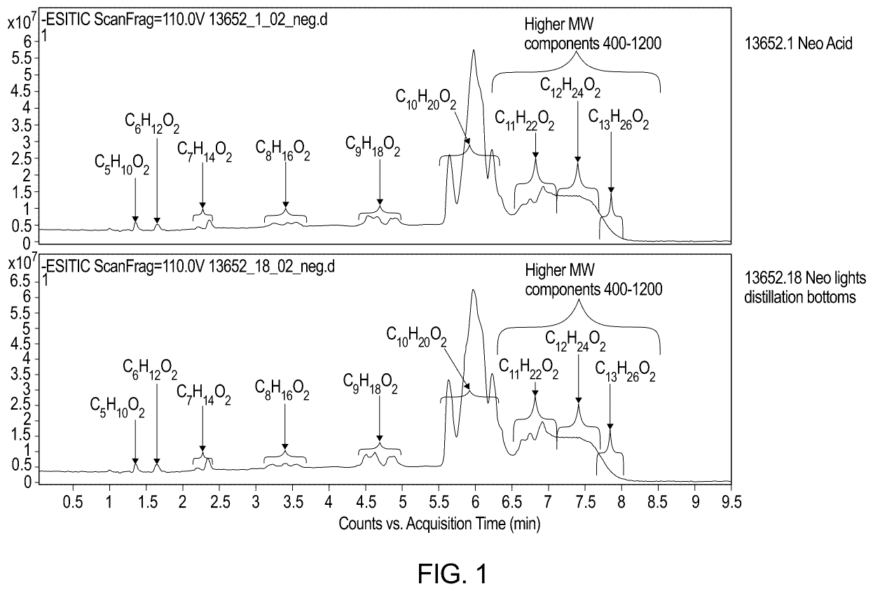

*LCMS analysis of the waste stream identified in preceding table, with hydrocarbons being identified in the instant table under “other high molecular weight products”
**LCMS analysis for two separate samples of the waste stream distilled to 100° C. included below in FIG. 1.

TABLE 3
Acid Number and Calculated Molecular Weight Comparison
of Neo Lights Stream at Various Stages of Purification

| | | | Post-Azeotropic |
|---|---|---|---|
| | Waste Stream | Post Distillation | distillation at |
| | as-received | to 100° C. | 95° C. |
| | (with | (most isoparaffins | (isoparaffins |
| | isoparaffins | removed, light | removed, light |
| Sample | and light acids) | acids still present) | acids removed) |
| Observed Acid | 252.1 | 257.7 | 378.4 |
| Number (mg | | | |
| KOH/g sample) | | | |
| Calculated Acid | 222.6 | 217.7 | 148.3 |
| Molecular Weight | | | |
| (g/mol) | | | |

In one embodiment, we have developed a process to isolate the useful carboxylic acids portion of this stream and react it with a metal source to produce a blend of metal carboxylate species which are anticipated to be effective H₂S scavenging chemicals. In one embodiment, we have intentionally utilized an “unrefined” or minimally refined synthetic acid stream in the production of a metal carboxylate-based H₂S scavenger, which is very attractive to do so because the resultant metal carboxylate product will have essentially identical hydrogen sulfide scavenging capabilities as an analogous “pure” metal carboxylate (i.e., a metal octoate/2-ethylhexanoate, neodecanoate, naphthenate, etc.) while retaining the requisite oil solubility and stability. This carboxylic acid starting material will have been produced at comparatively lower cost due to less processing time and energy associated with refinement to highly pure neodecanoic acid.

Additionally, in one embodiment, an unexpected benefit in utilizing the unrefined neodecanoic acid stream has been observed. When reacted with a molar excess of zinc oxide, the resultant metal carboxylate product appears to have a higher metal content than an analogous “pure” neodecanoate metal complex while maintaining a desirable fluidity and asphalt/oil solubility despite consisting of a range of carboxylic acids with carbon numbers ranging from 7 to 12, meaning the stream prepared from our process will actually perform better. Without being bound to any particular theory, in one embodiment, this appears to be because the unrefined acid component comprises a range of molecular weights which extends lower than C10, and the average MW is lower than that of pure neodecanoic acid.

In another embodiment, a crude neo lights stream is purified through an azeotropic distillation process by adding water to aid with the removal of the undesirable low flash isoparaffinic material and the majority of the lower weight carboxylic acids with carbon number in the range of C4-C6. The azeotropic distillation process comes with several added benefits: it reduces the thermal input necessary for the distillation as the azeotropic can be run at about 92-93° C. rather than >100° C., the azeotropic distillation very effectively removes the light isoparaffinic hydrocarbons without impacting the content of the desirable carboxylic acid content in the carbon number of 7-12 range, and also removes some of the lighter carboxylic acids with carbon number in the 4-6 range. Removal of the isoparaffin material increases the flash point of the resulting acid stream to a level acceptable for treatment of asphalt, and removal of the light acids decreases the tendency for crystalline metal complex formation in storage. For example, but without limitation, the flash point may be increased from 90° F. to 195° F., an increase of 105° F. In other embodiments, the flash point may be increased by 100° F. or more. The semi-purified acid mixture is then suitable to react with a metal source to produce a metal carboxylate which can then be used as a hydrogen sulfide scavenging additive for asphalt applications.

TABLE 4
Zinc content of Pure Zinc Neodecanoate vs.
Neo Lights Zinc Carboxylate from Bottoms

| Complex Identity | Molecular Formula Zinc Percentage |
|---|---|
| Zinc Neodecanoate (pure) | 20% |
| Zinc 2-Ethylhexanoate (pure) | 22.9% |
| Neo Lights Zinc Carboxylate (from | 22.4% |
| azeotropic bottoms) | |

The uses of the terms “a” and “an” and “the” and similar references in the context of the disclosure (especially in the context of the following claims) are to be construed to cover both the singular and the plural unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the foregoing description, the same is to be considered as illustrative and not restrictive in character, it being understood that all changes and modifications that come within the spirit of the invention are desired to be protected. In addition, all references cited herein are indicative of the level of skill in the art and are hereby incorporated by reference in their entirety.

### EMBODIMENTS

The following provides an enumerated listing of some of the embodiments disclosed herein. It will be understood that this listing is non-limiting, and that individual features or combinations of features (e.g., 2, 3 or 4 features) as described in the Detailed Description above can be incorporated with the below-listed Embodiments to provide additional disclosed embodiments herein.
1. A method of preparing an oil-soluble metal carboxylate, comprising reacting a metal oxide with a carboxylate stream, wherein the carboxylate stream comprises C5-C9 carboxylic acids, C10 carboxylic acids, and C11+ carboxylic acids.
2. The method of embodiment 1, wherein the C5-C9 carboxylic acids comprise about 0.1 to about 1 wt. % of the carboxylate stream.
3. The method of any one of the preceding embodiments, wherein the C11+ carboxylic acids comprise less than 10 wt. % of the carboxylate stream.
4. The method of any one of the preceding embodiments, wherein the C11+ carboxylic acids comprise about 5 to about 10 wt. % of the carboxylate stream.
5. The method of any one of the preceding embodiments, wherein the C10 carboxylic acids comprise about 75 to about 95 wt. % of the carboxylate stream.
6. The method of any one of the preceding embodiments, wherein the C5-C9 carboxylic acids comprise one or more acids selected from a C5 acid, a C6 acid, a C7 acid, a C8 acid, and a C9 acid.
7. The method of any one of the preceding embodiments, wherein the metal oxide comprises zinc oxide, an oxide of copper, or an oxide of iron.
8. The method of any one of the preceding embodiments, wherein the carboxylate stream further comprises non-acidic components.
9. The method of embodiment 8, wherein the non-acidic components comprise about 0.1 to about 5 wt. % of the carboxylate stream.
10. The method of embodiment 9, wherein the non-acidic components comprise about 0.5 to about 3 wt. % of the carboxylate stream.
11. The method of any one of embodiment 8-10, wherein the non-acidic components comprise at least one of aldehydes or ethers.
12. A method of preparing a high flash point mixed carboxylate stream comprising C5-C9 carboxylic acids, C10 carboxylic acids, and C11+ carboxylic acids from a mixture of said acids and low-flash isoparaffinic hydrocarbons.
13. The method of embodiment 12, wherein the high flash point mixed carboxylate stream is prepared by removing the low-flash isoparaffinic hydrocarbons by azeotropic codistillation with water which has been added to the distillation mixture.
14. The method of embodiment 12, wherein the high flash point mixed carboxylate stream is reacted with metal oxide with to form a metal mixed carboxylate stream, wherein the carboxylate stream comprises C5-C9 carboxylic acids, C10 carboxylic acids, and C11+ carboxylic acids.
15. The method of embodiment 14, wherein the C5-C9 carboxylic acids comprise about 0.1 to about 1 wt. % of the carboxylate stream.
16. The method of embodiment 14, wherein the C11+ carboxylic acids comprise less than 10 wt. % of the carboxylate stream.
17. The method of embodiment 14, wherein the C11+ carboxylic acids comprise about 5 to about 10 wt. % of the carboxylate stream.
18. The method of embodiment 14, wherein the C10 carboxylic acids comprise about 75 to about 95 wt. % of the carboxylate stream.
19. The method of embodiment 14, wherein the C5-C9 carboxylic acids comprise one or more acids selected from a C5 acid, a C6 acid, a C7 acid, a C8 acid, and a C9 acid.
20. The method of embodiment 14, wherein the metal oxide comprises zinc oxide, an oxide of copper, or an oxide of iron.

1. A method of preparing an oil-soluble metal carboxylate, comprising reacting a metal oxide with a carboxylate stream, wherein the carboxylate stream comprises C5-C9 carboxylic acids, C10 carboxylic acids, and C11+ carboxylic acids.

2. The method of claim 1, wherein the C5-C9 carboxylic acids comprise about 0.1 to about 1 wt. % of the carboxylate stream.

3. The method of claim 1, wherein the C11+ carboxylic acids comprise less than 10 wt. % of the carboxylate stream.

4. The method of claim 1, wherein the C11+ carboxylic acids comprise about 5 to about 10 wt. % of the carboxylate stream.

5. The method of claim 1, wherein the C10 carboxylic acids comprise about 75 to about 95 wt. % of the carboxylate stream.

6. The method of claim 1, wherein the C5-C9 carboxylic acids comprise one or more acids selected from a C5 acid, a C6 acid, a C7 acid, a C8 acid, and a C9 acid.

7. The method of claim 1, wherein the metal oxide comprises zinc oxide.

8. The method of claim 1, wherein the carboxylate stream further comprises non-acidic components.

9. The method of claim 8, wherein the non-acidic components comprise about 0.1 to about 5 wt. % of the carboxylate stream.

10. The method of claim 9, wherein the non-acidic components comprise about 0.5 to about 3 wt. % of the carboxylate stream.

11. The method of claim 8, wherein the non-acidic components comprise at least one of aldehydes or ethers.

12. A method of preparing a high flash point mixed carboxylate stream comprising C5-C9 carboxylic acids, C10 carboxylic acids, and C11+ carboxylic acids from a mixture of said acids and low-flash isoparaffinic hydrocarbons.

13. The method of claim 12, wherein the high flash point mixed carboxylate stream is prepared by removing the low-flash isoparaffinic hydrocarbons by azeotropic codistillation with water which has been added to the distillation mixture.

14. The method of claim 12, wherein the high flash point mixed carboxylate stream is reacted with metal oxide with to form a metal mixed carboxylate stream, wherein the carboxylate stream comprises C5-C9 carboxylic acids, C10 carboxylic acids, and C11+ carboxylic acids.

15. The method of claim 14, wherein the C5-C9 carboxylic acids comprise about 0.1 to about 1 wt. % of the carboxylate stream.

16. The method of claim 14, wherein the C11+ carboxylic acids comprise less than 10 wt. % of the carboxylate stream.

17. The method of claim 14, wherein the C11+ carboxylic acids comprise about 5 to about 10 wt. % of the carboxylate stream.

18. The method of claim 14, wherein the C10 carboxylic acids comprise about 75 to about 95 wt. % of the carboxylate stream.

19. The method of claim 14, wherein the C5-C9 carboxylic acids comprise one or more acids selected from a C5 acid, a C6 acid, a C7 acid, a C8 acid, and a C9 acid.

20. The method of claim 14, wherein the metal oxide comprises zinc oxide.
